(19) United States
(12) Patent Application Publication
(10) Pub. No.: US 2012/0271212 A1
(43) Pub. Date: Oct. 25, 2012

(54) LUMBAR SUPPORT BELT

(76) Inventor: Caren Grisham-Algots, Whittier, CA (US)

(21) Appl. No.: 13/455,703

(22) Filed: Apr. 25, 2012

Related U.S. Application Data
(60) Provisional application No. 61/517,810, filed on Apr. 25, 2011.

(51) Int. Cl. A61F 5/00

(52) U.S. Cl. 602/19

(57) ABSTRACT

The present invention provides a lumbar back support belt. A thin stretchable top layer is sewn over a thick stretchable bottom layer so that the thin layer is at the top viewable part of the belt and the thick layer is at the bottom part of the belt against the user. A pocket is sewn onto the top of the belt. A gripping mechanism is connected to the bottom part of the belt and is used for holding the belt in place around the user's waist. A hook and loop fastener is used for connecting the ends of the belt together. In a preferred embodiment, the gripping mechanism is a silicone strip. Also, in a preferred embodiment the hook and loop fastener is a Velcro® hook and loop fastener.

This application claims the benefit of Provisional Application No. 61/517,810 filed Apr. 25, 2011. The present invention relates to medical support devices, and in particular, to medical back support belts.

### BACKGROUND OF THE INVENTION

Lumbar back support belts are known. FIG. 1 shows prior art back support device 1. Back support device 1 is all black. It may be worn over the user's clothes or under the user's clothes. Belt 3 is attached via a Velcro® attachment mechanism 2. Velcro® is a registered trademark of Velcro Industries, Inc. and refers generally to a hook and loop fastening device. Belt 3 wraps around the waist of the user and fits snuggly to relieve pressure on the user's spine. To help hold the belt up, shoulder straps 4 are fitted over the user's shoulders. This helps to insure that the belt stays at the appropriate position around the user's waist.

Although prior art back support device 1 is effective in helping ease lower back pain and provide support to the lumbar spine, it is cumbersome and unattractive. Hence, patients are often hesitant to use device 1. Even though the device may help ease their pain, users may feel embarrassed due to the appearance of the device.

What is needed is a better lumbar support belt that has an attractive appearance.

### SUMMARY OF THE INVENTION

The present invention provides a lumbar back support belt. A thin stretchable visually pleasing top layer is sewn over a thick stretchable bottom layer so that the thin layer is at the top viewable part of the belt and the thick layer is at the bottom part of the belt against the user. A pocket is sewn onto the top of the belt. A gripping mechanism is connected to the bottom part of the belt and is used for holding the belt in place around the user's waist. A hook and loop fastener is used for connecting the ends of the belt together. In a preferred embodiment, the gripping mechanism is a silicone strip. Also, in a preferred embodiment the hook and loop fastener is a Velcro® hook and loop fastener.

### BRIEF DESCRIPTION OF THE DRAWINGS

FIG. 1 shows a prior art lumbar back support belt.

FIG. 2 shows a top view of a preferred embodiment of the present invention.

FIG. 3 shows a side view of the top layer and bottom layer of a preferred embodiment of the present invention.

FIG. 4 shows a top view of a preferred dart.

FIG. 5 shows a bottom view of a preferred embodiment of the present invention.

FIG. 6 shows a perspective view of a preferred embodiment of the present invention.

FIG. 7 shows a user wearing a preferred embodiment of the present invention.

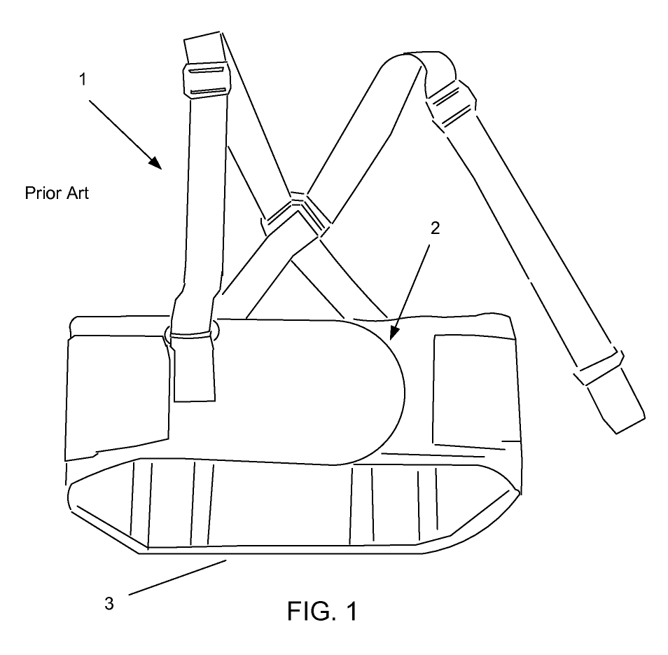

### DETAILED DESCRIPTION OF THE PREFERRED EMBODIMENTS

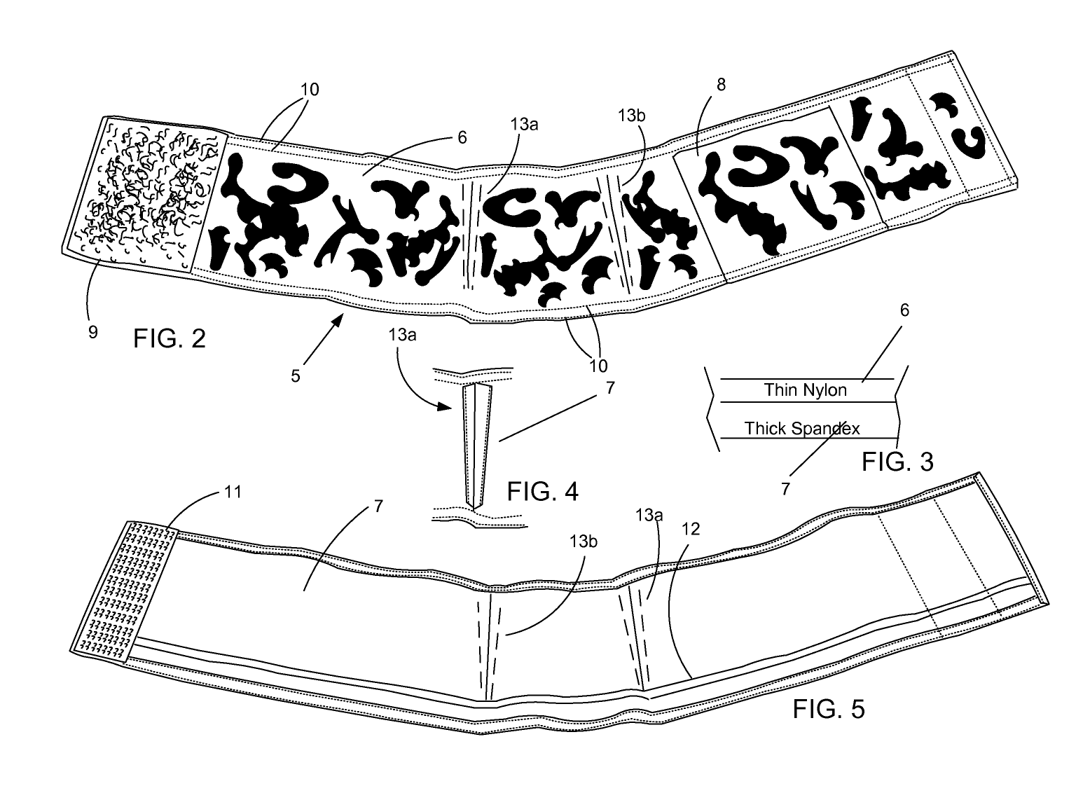

Lumbar support belt 5 (FIGS. 2, 5, 6 and 7) may be worn under the user's clothing or over the user's clothing. It includes an attractive outer layer so that the user is not embarrassed to wear the support belt. Belt 5 also includes a pocket for holding items of importance to the user, such as key, wallets or a cell phone.

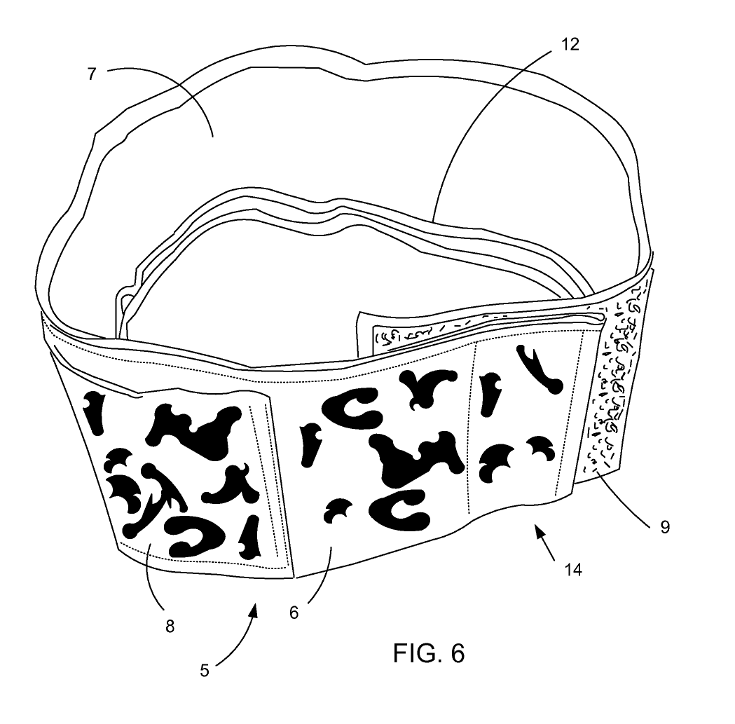

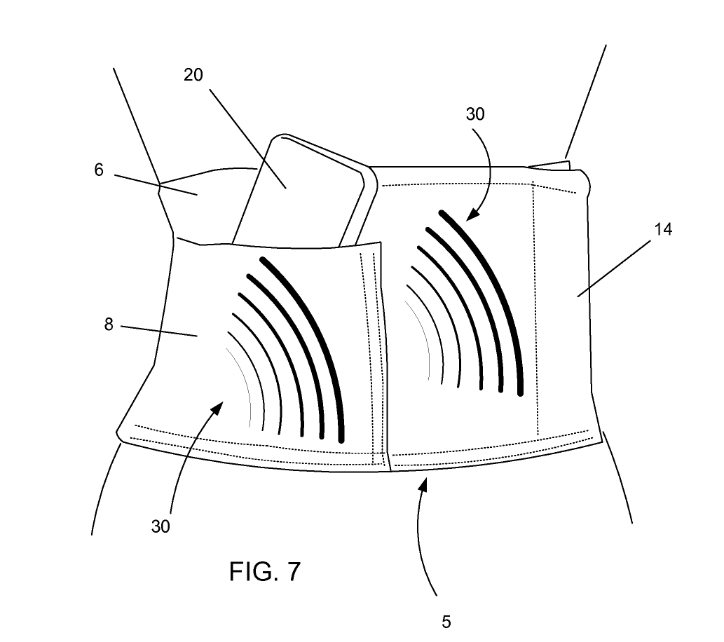

FIG. 2 shows a top view and FIG. 5 shows a bottom view of lumbar support belt 5. A stretchable outer visually pleasing thin layer 6 is sewn to a thick stretchable bottom layer 7 (FIG. 3). The primary purpose of the thin outer layer is to provide the decorative, visually pleasing appearance for belt 5. The primary purpose of the thick stretchable bottom layer 7 is to provide the strength and elasticity needed for belt 5 to function properly. Preferably, double stitches 10 are used to sew outer layer 6 to bottom layer 7. In a preferred embodiment thin outer layer 6 is stretchable nylon and thick stretchable bottom layer 7 is spandex. Pocket 8 is sewn on top of outer layer 6 as shown in FIG. 2. Loop section 9 is sewn to the top side of belt 5. Hook section 11 is sewn to the bottom side of belt 5. Together loop section 9 and hook section 11 form a Velcro® hook and loop fastener 14 (FIG. 6) for fastening the belt after the user has wrapped belt 5 around his waist. Heat set silicone strip 12 is glued to bottom layer 7 as shown in FIG. 5. Silicone strip 12 is a gripping mechanism that provides a gripping force so that belt 5 stays in the appropriate position around the user's waist while it is being worn. Silicone strip 12 will provide a comfortable gripping force against the user's clothing if belt 5 is worn outside the user's clothes or a comfortable gripping force against the user's skin if worn underneath the user's clothes. Silicone strip 12 keeps belt 5 from moving upwards or downwards while the belt is being worn. Darts 13a and 13b are sewn into bottom layer 7 to add strength, shape and shape to bottom layer 7. FIG. 4 shows a top view of bottom layer 7 with outer thin layer 6 removed to present to provide a more detailed view of dart 13a.

FIG. 6 shows a perspective view of belt 5. Belt 5 is fastened together utilizing hook and loop fastener 14. As stated above, after belt 5 is fastened around the waist of the user silicone strip 12 provides friction force that will help prevent belt 5 from moving upward or downward. Items may also be placed inside pocket 8 as desired by the user.

FIG. 7 shows a user wearing a preferred belt 5. Belt 5 has been fastened utilizing hook and loop fastener 17. Cell phone 20 has been placed inside pocket 8 for easy access. Decorative pattern 30 is displayed on outer decorative thin layer 6 and pocket 8 as shown.

Although the above-preferred embodiments have been described with specificity, persons skilled in this art will recognize that many changes to the specific embodiments disclosed above could be made without departing from the spirit of the invention. For example, although the above preferred embodiments showed specific decorative patterns, it is possible to substitute a variety of decorative patterns onto outer decorative thin layer 6 and pocket 8. It is also possible for outer decorative thin layer 6 to be a solid attractive color (such as purple, green or red). Therefore, the attached claims and their legal equivalents should determine the scope of the invention.

What is claimed is:

1) A lumber back support belt, comprising:
A) a thick stretchable bottom layer,
B) a thin stretchable top layer sewn to said bottom layer, wherein said thin layer forms the top of said belt and wherein said thick layer forms the bottom of said belt,
C) at least one pocket sewn onto said top of said belt,
D) a gripping mechanism for gripping the user's clothes or the user's skin and for holding said belt in place around the user's waist, and
E) a hook and loop fastener for connecting the ends of said belt.

2) The lumbar back support belt, as in claim 1, wherein said thin stretchable top layer is nylon.

3) The lumbar back support belt as in claim 1, wherein said thin stretchable top layer comprises a decorative, visually pleasing design to improve the appearance of said belt.

4) The lumbar back support belt as in claim 1, wherein said thick stretchable bottom layer is spandex.

5) The lumbar back support belt as in claim 1, wherein darts are sewn into said thick stretchable bottom layer to provide strength, shape and support to said belt.

6) The lumbar back support belt as in claim 1, wherein said gripping mechanism is a silicone strip.

7) The lumbar back support belt as in claim 1, wherein said hook and loop fastener is a Velcro® hook and loop fastener.
